(19) United States
(12) Patent Application Publication
(10) Pub. No.: US 2023/0381213 A1
(43) Pub. Date: Nov. 30, 2023

(54) SYNTHESIS AND USES OF GINSENOSIDE COMPOUND K DERIVATIVES

(71) Applicants: The University of Hong Kong, Hong Kong (CN); Laboratory for Synthetic Chemistry and Chemical Biology Limited, Hong Kong (CN)

(72) Inventors: Chi-Ming CHE, Hong Kong (CN); Yungen LIU, Hong Kong (CN); Jilin HE, Hong Kong (CN); Chun-Nam LOK, Hong Kong (CN)

(21) Appl. No.: 18/315,937

(22) Filed: May 11, 2023

Related U.S. Application Data
(60) Provisional application No. 63/365,528, filed on May 31, 2022.

(51) Int. Cl. A61K 31/704; A61P 35/00

(52) U.S. Cl. CPC A61K 31/704; A61P 35/00

(57) ABSTRACT

A nontoxic anticancer compound is a derivative of 20-O-β-(D-glucopyranosyl)-20(S)-protopanaxadiol (CK) having at least one of the glucose hydroxyl groups replaced with at least one acetal group. The derivative enhances binding to a mitochondrial membrane protein and is more cytotoxic to cancerous cells than CK. The derivative can be an acetal of an unsubstituted or substituted aromatic group, such as an acetal formed from a substituted 1-(dimethoxymethyl)-benzene. One or more of the CK derivative (CKD) anticancer compounds can be used as an active portion of an anticancer medicament.

### CROSS-REFERENCE TO RELATED APPLICATION

This application claims the benefit of U.S. Provisional Application Ser. No. 63/365,528, filed May 31, 2022, which is hereby incorporated by reference in its entirety including any tables, figures, or drawings.

### BACKGROUND OF THE INVENTION

Lung cancer is one of the most common cancer types and the leading cause of cancer associated death worldwide. Non-small-cell lung cancer (NSCLC) accounts for around 85% of the diagnosed lung cancer and has poor therapeutic efficacy. Ginseng has been used for thousands of years in Asian countries as traditional medicine for replenishing vital energy without side effects. The main components of ginseng are ginsenosides which possess a steroidal core containing four trans-rings with a modified side chain at C-20 and saccharide chains at different positions. Compound K (CK) (20-O-β-(D-glucopyranosyl)-20(S)-protopanaxadiol) is the intestinal bacterial metabolite of ginsenosides Rb1, Rb2, and Rc. Over the decades, numerous studies about the anti-cancer activity of CK in various cancer cell lines including lung cancer, liver cancer, breast cancer and colorectal cancer have been described. CK suppressed the proliferation of lung cancer cells including NCI-H460, A549 and H1299 via hypoxia-inducible factor-1α (HIF-1α) regulated glucose metabolism by inhibition of expression and the downstream gene GLUT1. CK induces apoptosis and autophagy in A549 and H1975 cells through AMPK-mTOR and JNK pathways. CK has synergistic effects with other therapeutics. For instance, efficacy of cisplatin improves when co-treatment with CK in lung cancer and the effect is p53 dependent. Combination therapy of CK with γ-ray radiation is effective in both cell culture and NCI-H460 tumor xenografts models. Nevertheless, the molecular targets of CK in lung cancer remains obscure.

Different chemical proteomics methods have been developed for target identification including activity-based protein profiling (ABPP), thermal proteome profiling (TPP) and functional identification of target by expression proteomics (FITExP). The principle of TPP is based on ligand binding inducing thermal stability of target proteins. A western blot format called cellular thermal shift assay (CETSA) has been designed based on this phenomenon to validate the drug and target interaction. Savitski et al. extended CETSA in a proteome wide manner using liquid chromatography with tandem mass spectrometry (LC-MS/MS) so that the change of thousands of proteins induced by drug treatment could be monitored simultaneously. A simplified version of TPP called proteome-wide integral solubility alteration (PISA) was proposed by Zubarev's group where the difference of Sm (the integral of the area under the melting curve of each protein) of each protein between different samples is measured. In contrast, ProTargetMiner (PTM), is a proteome signature database of anticancer compounds (>50 drugs) based on FITExP. Similar to FITExP, PTM monitors change of protein expression after long-term (normally 48 h) incubation with drugs in cells at a LC50 concentration. Both methods are based on the phenomenon that protein targets show strong and specific modulation upon drug stimulation. To have a more specific target information, PTM introduces orthogonal projections to a latent structures discriminant analysis (OPLS-DA) in comparison with other anticancer drugs in the database (http://proteargetminer.genexplain.com).

Mitochondria contributes to the production of ATP, macromolecules, apoptosis, and oxidative stress. Mitochondria are involved in key steps of cancer aggressiveness, sustaining tumor growth and cancer progression. Mitochondrial membranes are made up of different phospholipids such as cardiolipin (CL) and phosphatidylethanolamine (PE). Cardiolipins (CLs) are exclusive to mitochondria and associate with mitochondrial dynamics and are involved in the oxidative phosphorylation system, maintaining mitochondrial cristae organization. Dysregulation of CL metabolism is essential in anti-cancer therapy of different types of cancer. It is important to maintain the composition, transport, and membrane allocation of these phospholipids for mitochondrial homoeostasis. The proteins of relevant evolutionary and lymphoid interest (PRELI) domain containing family are responsible for the lipid transfer systems in mitochondria. This family is conserved from yeast system across all eukaryotic life, highlighting its importance in cellular viability via maintaining mitochondrial lipid homeostasis, and has an important role in malignant cancers. Knock down of PRELID1 suppressed breast cancer cell growth and associated with the cell apoptosis in liver cancer. There is no direct relationship between PRELID3b and cancer, however, knock down of PRELID3b in Hela cell inhibits expression of PRELID1 and mitochondrial Dynamin Like GTPase (OPA1), where both of these two proteins play important roles in mitochondrial function.

The mechanism(s) of anti-cancer activity of ginsenosides remain(s) unclear. One of the reasons for this is the complex structure of ginsenosides, which are challenging in transformation into derivatives. Although CK has been shown to exert anticancer activity in lung cancer cells, it's in vivo effect is not potent, mainly attributed to its low bioavailability. Modifications of CK have not resulted in enhanced anti-cancer activity. Hence the modification of CK that achieves a greater activity is desirable.

### BRIEF SUMMARY OF THE INVENTION

Embodiments of the invention are directed to an anticancer compound that is a derivative of 20-O-β-(D-glucopyranosyl)-20(S)-protopanaxadiol (CK), where at least one of the glucose hydroxyl groups has been converted into at least one acetal group. The acetal group provides a hydrophobic portion that enhances binding between the anticancer compound and a mitochondrial intermembrane protein that is capable of assembling a lipid transfer complex with TRIAP1 in mitochondria. One of the CK derivatives (CKDs), CKD-4 displays higher cytotoxicity than CK.

In embodiments the CKD can be an acetal derivative with the structure:

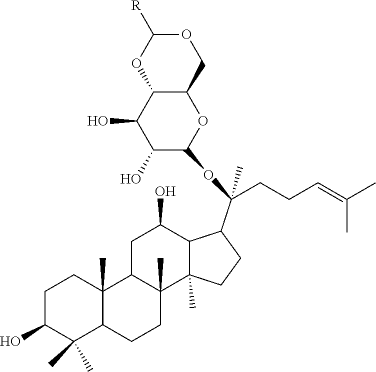

where R is an unsubstituted, monosubstituted, or disubstituted aromatic group. For example, but not limited to, R can be —C₆HₓX₅₋ₓ where x is 3 to 5 and X is independently selected from methoxy, nitro, trifluoromethyl or trifluoromethoxy where X substituents can be in any of the para, meta, or ortho position to the acetal. The R can be p-methoxyphenyl, phenyl, o-methoxyphenyl, m-methoxyphenyl, p-nitrophenyl, p-trifluoromethyl or p-trifluoromethoxyphenyl. Other acetal comprising CKDs can form acetals of 2,2-propane or 2,2-fluoromethane with two adjacent glucose hydroxyl groups.

In embodiments, one or more of the anticancer compounds described above can be included in an anticancer medicament. These CKD compounds can be used as an alternative or in conjunction with currently employed cancer drugs.

Other embodiments are directed to methods of preparing an anticancer compound from CK, where CK is combined in a reaction mixture with an aldehyde, acetal, ketone, or ketal to form the CKD. The CKD is then isolated by common isolation methods, which may include crystallization, distillation, precipitation, or chromatographic methods. The reagents can be combined in a solvent and a catalyst can be included in the reaction mixture.

### BRIEF DESCRIPTION OF THE DRAWINGS

FIG. 1 shows the chemical structure of CK and five CK derivatives (CKDs), according to embodiments.

FIG. 2A shows a structural equation for the synthesis of CKD-2, according to an embodiment, where a is 2,2-dimethoxy propane:acetone (1:5) and p-toluenesulfonic acid.

FIG. 2B shows a structural equation for the synthesis of CKD-3, where b is 2,2-dimethoxy propane and p-toluenesulfonic acid.

FIG. 2C shows a structural equation for the synthesis of CKD-4 and CKD-5, according to an embodiment, where c is boron trifluoride diethyl etherate in dry dichloromethane.

FIG. 3 shows a bar chart for CK and CKD-4 uptake in A549 cells treated with 10 μM of CK or CKD-4 after 4 and 24 hours (n=3 biological independent replicates).

FIG. 4 shows flow cytometry plots for on A549 cells treated with 60 μM of CK or 20 μM of CKD-4 for 24 hours, and collected and stained with Annexin V-FITC and Propodium iodide.

FIG. 5 shows microscopic 100× bright field images of control and CK or CKD-4 treated MS 1 cells seeded on a Matrigel pre-coated 96-well-plate where cells were treated with various concentration of CK or CKD-4 for 4 hours.

FIG. 6 shows images of lung cancer derived organoids after treatment with DMSO, caroboplatin, cisplatin, paclitaxel and 5, 10, 20, 35, and 50 μM concentrations of CKD-4.

FIG. 7 shows a bar chart of the CKD-4 inhibits proliferation of patient derived organoids of FIG. 7 using CellTiterGlo™ at 72 hours where cell viability of DMSO treatment was regarded as 100% (control) plotted as mean±SD.

FIG. 8A shows a plot of the changes in tumor volume for in vivo cytotoxicity upon treatment with 5 mg/kg of CK and 5 mg/kg of CKD-4 with data as mean±SEM. **, P<0.01, *, P<0.05.

FIG. 8B shows a plot of the changes of body weight volume of mice upon treatment with 5 mg/kg of CK and 5 mg/kg of CKD-4.

FIG. 9A shows a volcano plot of PISA analysis of CK in cell.

FIG. 9B shows a volcano plot of PISA analysis of CKD-4 in cell.

FIG. 10 shows a plot of the OPLS-DA analysis for CK versus 9 drugs and controls in A549 cells where the top 20 upregulated proteins with the top 20 upregulated proteins labelled in red and top 20 downregulated proteins labelled in blue and where RELID3b is labelled as green.

FIG. 11 shows a plot of the OPLS-DA analysis for CKD-4 versus 9 drugs and controls in A549 cells where the top 20 upregulated proteins with the top 20 upregulated proteins labelled in red and top 20 downregulated proteins labelled in blue and where RELID3b is labelled as green.

FIG. 12A is a scatter plot of protein expression fold change for CKD-4 relative to DMSO treated cells vs CK relative to DMSO treated cells in PTM.

FIG. 12B is a scatter plot of protein expression fold change for CK relative to DMSO treated cells vs methotrexate relative to DMSO treated cells in PTM.

FIG. 12C is a scatter plot of protein expression fold change for CKD-4 relative to DMSO treated cells vs methotrexate relative to DMSO treated cells in PTM.

FIG. 13A is a plot of the cumulative ranking of fold changes by PISA and PTM analysis in the presence of CK.

FIG. 13B is a plot of the cumulative ranking of fold changes by PISA and PTM analysis in the presence of CKD-4.

FIG. 14A shows the cellular thermal shift assay of PRELID3b upon treatment with CK or CKD-4 in A549 cells. A549 cells were treated with 50 μM of CK or 15 uM of CKD-4 for one hour, where eight lysate aliquots were heated to indicated temperature.

FIG. 14B shows the cellular thermal shift assay of PRELID1 upon treatment with CK or CKD-4 in A549 cells. A549 cells were treated with 50 μM of CK or 15 μM of CKD-4 for one hour, where eight lysate aliquots were heated to indicated temperature.

FIG. 15A is a bar chart of the CK and CKD-4 induced apoptosis by quantification of cardiolipin in mitochondrial after treatment with CK or CKD-4 for 24 hours in A549 cells as mean±SD, **, P≤0.01, *, P≤0.05.

FIG. 15B shows western blotting analysis of cytosol fractions of cytochrome c after treatment with 50 μM of CK and 15 μM of CKD-4 for 24 and 48 hours with COX IV indicating no mitochondrial contamination.

FIG. 15C is a bar chart of the cell viability of A549 after treatment with 100 μM of CK or 12.5 μM of CKD-4 for 24 hours in DMEM or phosphatidylglycerol (PG) supplemented DMEM as mean±SD, ***P≤0.001.

FIG. 15D shows western blotting analysis of cytosol fractions of cytochrome c after treatment with 50 μM of CK and 15 μM of CKD-4 for 24 hours with PG or without PG in medium with COX IV indicating no mitochondrial contamination.

FIG. 16A shows computer generated image for the docked pose of CK with PRELID3b with the surface of the protein representing the electrostatic potential surface with red to blue infers a change of potential from negative to positive and the interactions between CK and surrounding residues.

FIG. 16B shows computer generated image for the interaction of CK with the amino acid residues of the binding site of PRELID3b.

FIG. 16C is a plot of the rmsd profiles of CK during 50 ns MD simulations.

FIG. 16D shows computer generated image for the docked pose of CKD-4 with PRELID3b with the surface of the protein representing the electrostatic potential surface with red to blue infers a change of potential from negative to positive and the interactions between CKD-4 and surrounding residues.

FIG. 16E shows computer generated image for the interaction of CKD-4 with the amino acid residues of the binding site of PRELID3b.

FIG. 16F is a plot of the rmsd profiles of CKD-4 during 50 ns MD simulations.

FIG. 17A shows biolayer interferometry binding curves using his tag PRELID3b-TRIAP1-MBP and CK of various concentrations.

FIG. 17B shows plots for Kd calculated by steady-state analysis assuming the kinetics as 1:1 ratio of his tag PRELID3b-TRIAP1-MBP to CK.

FIG. 17C shows biolayer interferometry binding curves using his tag PRELID3b-TRIAP1-MBP and CKD-4 of various concentrations.

FIG. 17D shows plots for Kd calculated by steady-state analysis assuming the kinetics as 1:1 ratio of his tag PRELID3b-TRIAP1-MBP to CKD-4.

FIG. 17E shows biolayer interferometry binding curves using his tag MBP and CK of various concentrations.

FIG. 17F shows biolayer interferometry binding curves using his tag MBP and CKD-4 of various concentrations.

### DETAILED DISCLOSURE OF THE INVENTION

In embodiments, derivatives of the ginsenoside, 20-O-β-(D-glucopyranosyl)-20(S)-protopanaxadiol (CKDs) are prepared that improve CK activity for anticancer compositions. The derivatives have various numbers of protected glucose hydroxyl groups. One derivative, CKD-4, according to an embodiment, has a higher cellular uptake than CK in A549 cells and display a much greater cytotoxicity than CK in vitro and in vivo. The molecular target of CK and CKD-4 is PRELID3b, suggesting a significant relationship between PRELID3b and cancer. Anticancer activity of ginsenosides increases with decreasing number of saccharides, where ginsenosides with one moiety of saccharide has the strongest cytotoxicity, such as Rh2 and CK, and the glucose moiety in CK is essential to its interaction with its molecular targets. Both CK and CKD-4 interact with PRELID3b and appear to bind to the lipid binding pocket of the protein. CKD-4 has stronger anti-cancer activity than CK both in vitro and in vivo. The mechanism of CK and CKD-4 is similar as both compounds engage the mitochondrial inter membrane proteins, PRELID3b and PRELID1. Based on these interactions, the two compounds appeared to disrupt the normal transportation of phospholipids, leading to a reduction in mitochondrial levels of CL. The treatment with these anti-cancer compounds facilitates the release of cytochrome c and mitochondrial fission, leading to mitochondrial dysfunction and apoptosis.

The novel anticancer compounds, according to embodiments of the invention, are formed by the modification of natural products and the study of their new mode of action (MOA). New and potent CK derivatives, such as, but not limited to CKD-4, exhibit stronger cytotoxicity and higher inhibition rate in animal models than the natural product from which they are derived, and display relatively low toxicity. Using a combined proteomics approach with proteome-wide integral solubility alteration (PISA) and ProTargetMiner (PTM)), identified the molecular target, PRELID3b, for CK, CKD-4, and other CK derivatives of CK, that resides in the lung cancer cell line. PRELI-like family proteins regulate lipid metabolism in mitochondria and emerging evidence shows their importance in tumor progression. The new anticancer treatment involves a novel strategy of treatment, that, according to embodiments, target PRELID3b.

The 20-O-β-(D-glucopyranosyl)-20(S)-protopanaxadiol (CK) derivatives are produced by reaction with one or more hydroxy group of the monosaccharide unit of CK:

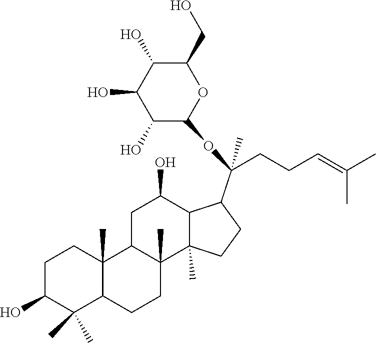

(CK). The derivatization can be performed at any single hydroxy group or any plurality of or hydroxy groups. The plurality of hydroxy groups can be on adjacent carbons or may be separated by a chain of two or more carbons. In one embodiment, two hydroxy groups form an acetal or a ketal upon derivatization. The derivatives provide a molecular portion that can provide an additional hydrophobic portion of the molecule, which can provide an advantageous balance of hydrogen bonding and van der Waal interactions with portions of the proteins targeted by the anticancer agent.

In one embodiment the anticancer compound is an aromatic acetal derivative of CK having the structure:

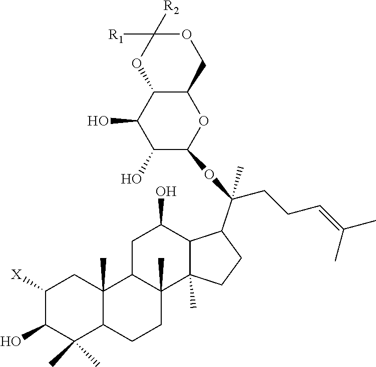

(CKD-x). X is H, R₁ is independently H, F or methyl and R₂ is independently F, methyl or an unsubstituted, monosubstituted, or disubstituted aromatic group. Substituents of a monosubstituted, or disubstituted phenyl or other aromatic group can be, but not limited to, methoxy, nitro, trifluoromethyl, or trifluoromethoxy where one or more of these substituents can be present in the ortho, meta, or para positions relative to the saccharide ring. A non-exhaustive list of CKDs includes, CKD-2 (2, 2-dimethyl), CKD-4 (p-methoxyphenyl), CKD-5 (phenyl), CKD-6 (o-methoxyphenyl), CKD-7 (m-methoxyphenyl), CKD-8 (p-nitrophenyl), CKD-9 (p-trifluoromethylphenyl), CKD-10 (p-trifluoromethoxyphenyl) and CKD-11 (2, 2-difluoro).

In other embodiments the CKD-x can be a compound of the structure:

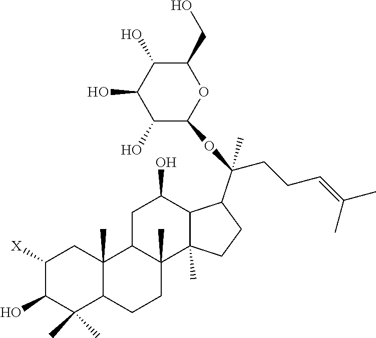

(CKD-x). Substituent X can be F or other halogen, for example:

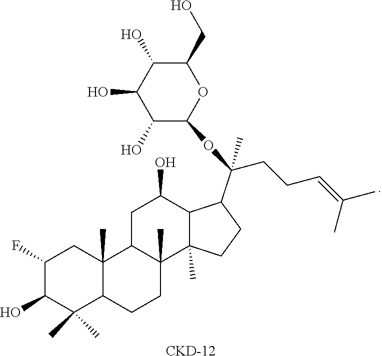

Both PRELID3b and PRELID1 belong to the same family that assembles into a lipid transfer complex with TRIAP1 in mitochondria. Specifically, PRELID3b/TRIAP1 carries PS from endoplasmic reticulum (ER) to mitochondrial inner membrane (IM) for synthesis of PE and PRELID1/TRIAP1 transports PA across the intermembrane space (IMS) for synthesis of CL. Therefore, the interaction/engagement of CK or CKD-4 with these two complexes appear to cause change accumulation of CL and PE. Although the two complexes specifically transport the precursor of PE or CL, either the deletion of PRELID1 or PRELID3b in Hela cells affect the accumulation of both CL and PE. A decrease in the level of CL in A549 cells occurs after treatment with CK or CKD-4. This reduction of CL triggers release of cytochrome c from mitochondria, results in cell apoptosis. Cytochrome c release from mitochondria during CK or CKD-4 induced cell apoptosis that is restored after the recovery of CL level by replenishment of its precursor, phosphatidylglycerol (PG). Moreover, the inhibition of cell viability by CK or CKD-4 also suppressed after supplement of PG.

Cytotoxicity of CK was measured using MTT assay towards various cancer cell lines, as shown in Table 1, below. CK is cytotoxic towards lung cancer (NCI-H460), cervical cancer (Hela), liver cancer (MHCC97-L and HepG2) and colon cancer (SW480) at sub-micro molar levels without selectivity.

TABLE 1
Cytotoxicity (IC₅₀, μM) of CK in various cancer cell lines for 72 hours.

| | NCI-H460 | Hela | MHCC97-L | HepG2 | SW480 |
|---|---|---|---|---|---|
| | Lung | Cervical | Liver | Liver | Colon |
| | cancer | cancer | cancer | cancer | cancer |
| CK | 45.1 ± 0.4 | 36.2 ± 1.2 | 49.8 ± 0.3 | 43.6 ± 1.6 | 46.2 ± 2.1 |

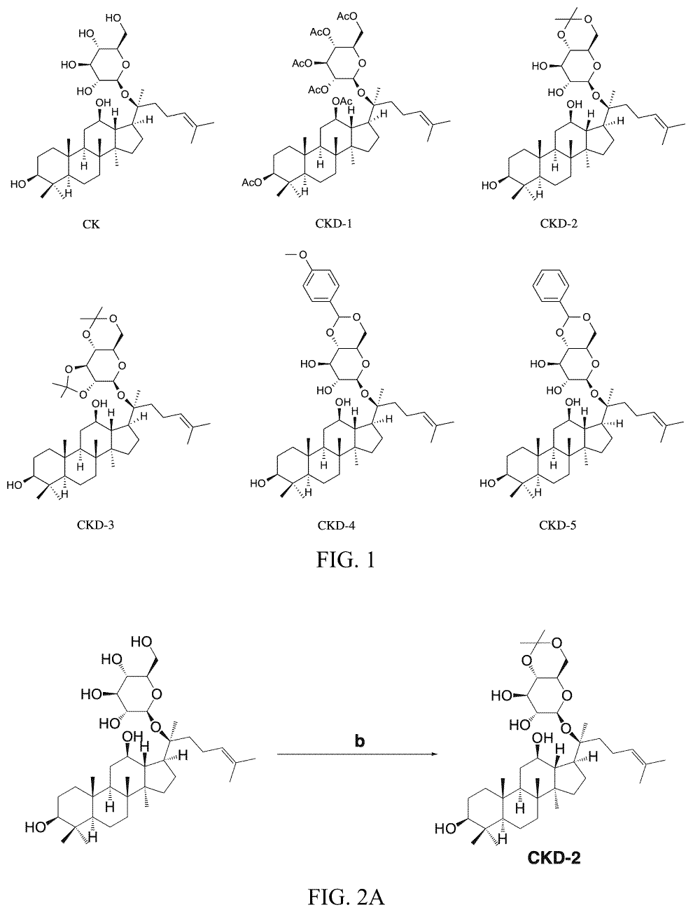

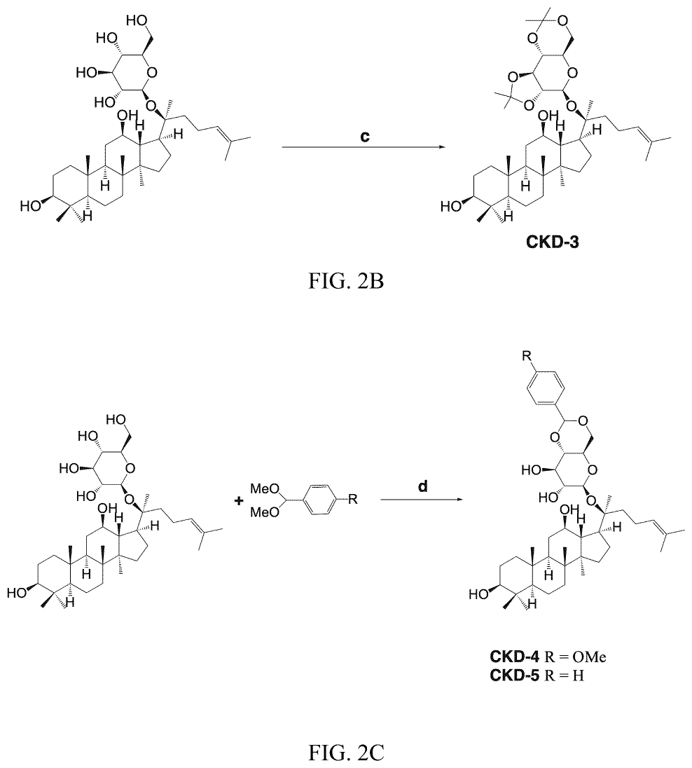

Five CK derivatives were synthesized by protecting various number of hydroxyl groups, as shown in FIG. 1. CKD-1 was synthesized by acetylation of all the six hydroxyl groups. Synthesis of derivatives CKD-2, CKD-3, CKD-4, and CKD-5 are shown in FIG. 2A through 2C. CKD-1 is highly hydrophobic and has poor solubility, even in dimethyl sulfoxide (DMSO). The other four derivatives were synthesized by protecting the hydroxyl groups via cyclic acetal formation. The cytotoxicity of these four compounds was measured by MTT assay in NCI-H460 cells and shown in Table 2, below. Among the five newly synthesized compounds, CKD-4 was the most potent one with the IC₅₀ value at 16.5 μM. The IC₅₀ value of CKD-2 was almost the same with CK while CKD-3 lost its anti-cancer activity. Besides, the cytotoxicity of CKD-4 was further validated in other lung cancer cell lines as well as lung normal fibroblast (CCD-19Lu). In comparison with CK, CKD-4 was three-fold more potent although both of them showed no difference between cancer cell lines and lung normal fibroblasts, as indicated in Table 3, below.

TABLE 2
Cytotoxicity (IC₅₀, μM) of CK and its derivatives in NCI-H460 cells
treated with CK and CKD-x derivatives for 48 h.

| | IC₅₀ |
|---|---|
| CK | 53.4 ± 2.4 |
| CKD-1 | >100 |
| CKD-2 | 39.1 ± 0.9 |
| CKD-3 | >100 |
| CKD-4 | 16.5 ± 0.1 |
| CKD-5 | 25.3 ± 0.2 |

TABLE 3
Cytotoxicity (IC₅₀, μM) of CK and CKD-4 in different lung
cancer cell lines and lung normal fibroblast for 24, 48 and 72 h.

| | IC₅₀ 24 h | | IC₅₀ 48 h | | IC₅₀ 72 h | |
|---|---|---|---|---|---|---|
| | CKD-4 | CK | CKD-4 | CK | CKD-4 | CK |
| NCI-H460 | 23.3 ± 1.4 | 64.7 ± 9.3 | 16.5 ± 0.1 | 53.4 ± 2.4 | 15.4 ± 0.1 | 40.5 ± 0.9 |
| A549 | 30.8 ± 3.7 | 67.1 ± 4.6 | 23.9 ± 0.9 | 64.0 ± 3.3 | 17.4 ± 1.2 | 59.1 ± 3.4 |
| NCI-H1650 | 43.7 ± 4.4 | 80.7 ± 3.7 | 25.1 ± 1.5 | 71.3 ± 7.1 | 20.3 ± 1.1 | 59.3 ± 2.8 |
| CCD-19Lu | 57.5 ± 0.6 | 73.6 ± 3.4 | 25.9 ± 4.4 | 63.1 ± 2.7 | 17.9 ± 2.8 | 42.3 ± 1.5 |

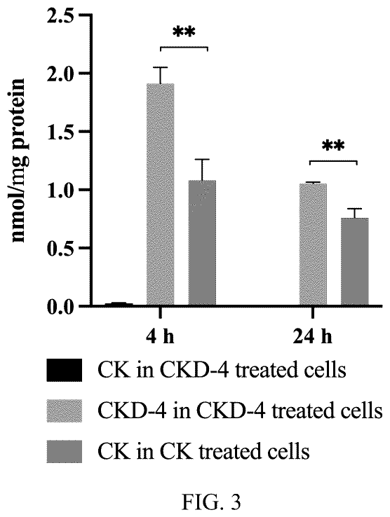

Cellular uptake of CK and CKD-4 A549 cells monitored at two different incubation time duration (4 and 24 hours), is shown in FIG. 3, where CKD-4 has significantly higher uptake than CK for both periods. This suggests that the improvement in cytotoxicity of CKD-4 maybe attributed by the increased cellular uptake compared to CK. To explore the stability of CKD-4, the amount of CK in CKD-4 treated A549 cells was also quantified. As shown in FIG. 3, only negligible amount of CK (1.33% of total amount of CKD-4 at 4 h and 1.05% of total amount of CKD-4 at 24 h) was found in cells. This was significantly lower than the amount of CKD-4 in CKD-4 treated cells, suggesting that CKD-4 is the main functional component in CKD-4 treated cells rather than CK.

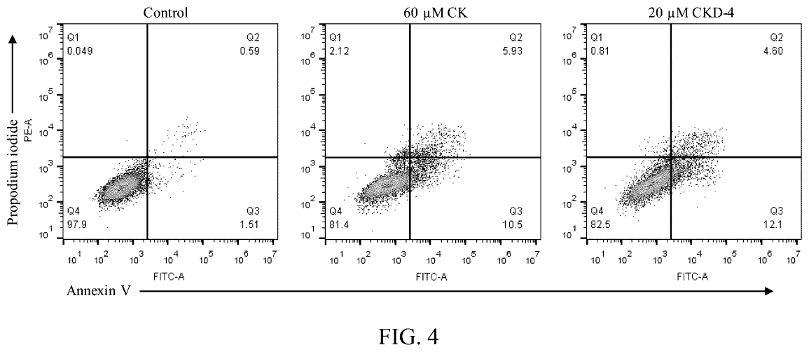

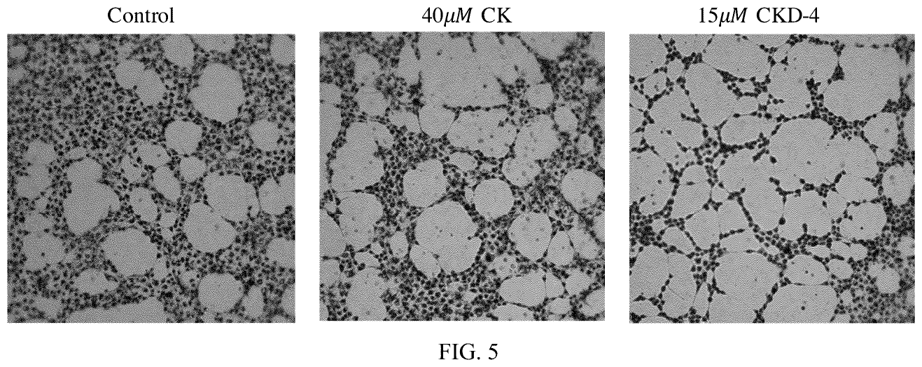

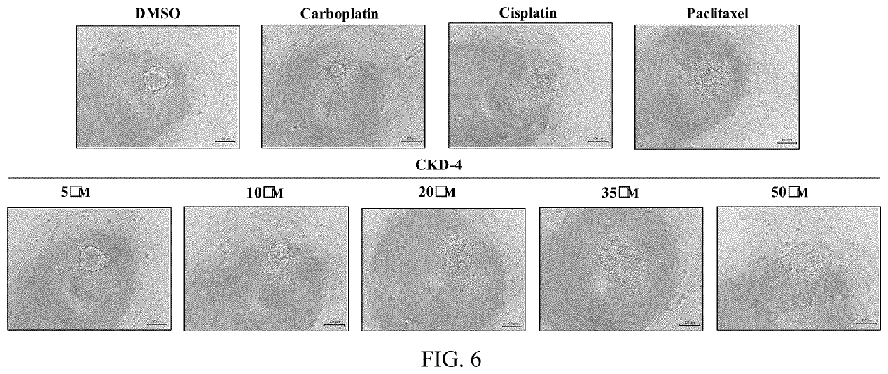

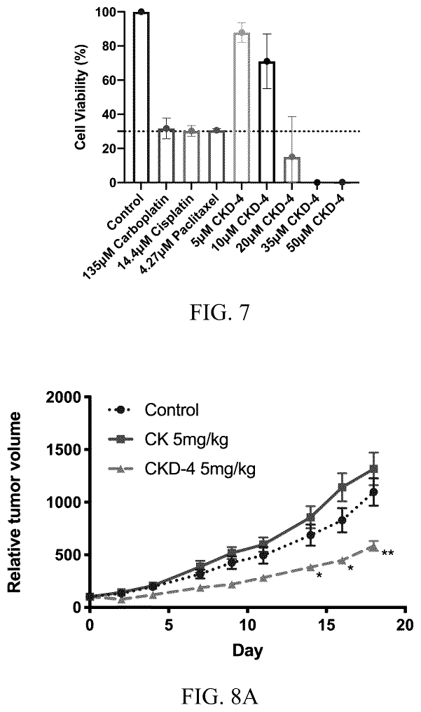

The effect of CK and CKD-4 on cell apoptosis and cell death is indicated by A549 cells treated with 60 μM of CK and 20 μM of CKD-4 for 48 hours that are analyzed by flow cytometry. FIG. 4 indicates that for CK and CKD-4 there is a significant increase in the percentage of apoptotic cells. Angiogenesis is another hallmark of cancer which plays an important role in metastasis and tumor growth. In comparison with the untreated MS 1 cells, the formation of tube is inhibited by 40 μM of CK while the inhibition is more obvious when treated by 15 μM of CKD-4, as indicated in FIG. 5. Cancer stem cells, owing to their ability of self-renewal, have become the major reason for relapse of tumor and poor prognosis. The efficacy of CKD-4 in lung cancer patient derived organoid is indicated using CellTiterGlo assay, where three state of the art clinical anti-cancer drugs, carboplatin, cisplatin, and paclitaxel are a positive control. The dosage of these three drugs is sufficient to trigger a 30% inhibition of cell viability. FIG. 6 and FIG. 7 indicated less than 30% suppression of cell viability is observed with treatment of 20 μM CKD-4. As depicted in FIG. 7, CKD-4 significantly inhibits the growth of patient derived organoid in a dose dependent manner.

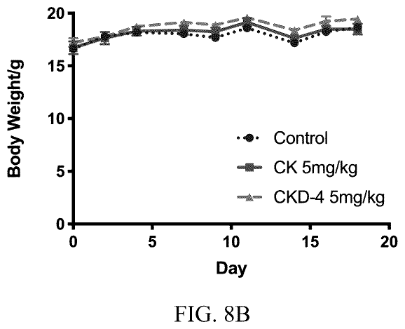

Treatment of nude mice bearing NCI-H460 xenografts with 5 mg/kg of CK and CKD-4 through intravenous once every two days, shows significant tumor volume inhibition in the group of CKD-4 while almost no difference is observed between the group of CK and solvent control, as shown in FIG. 8A. No significant change in body weight is observed throughout the treatment, as indicated in FIG. 8B. Hence, CKD-4 is more potent than CK by in vitro and in vivo.

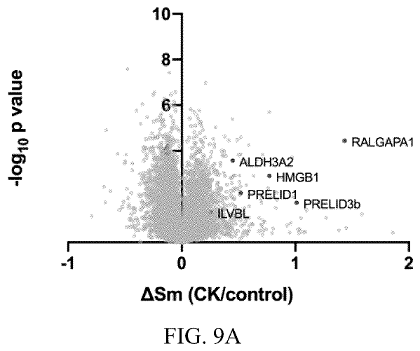

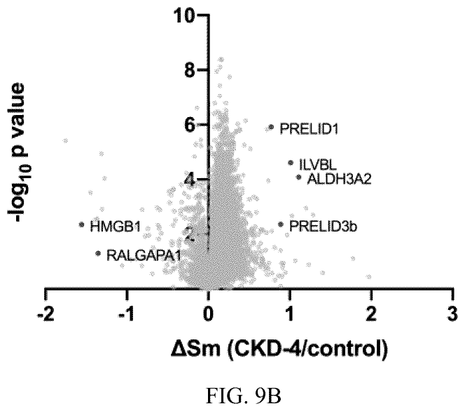

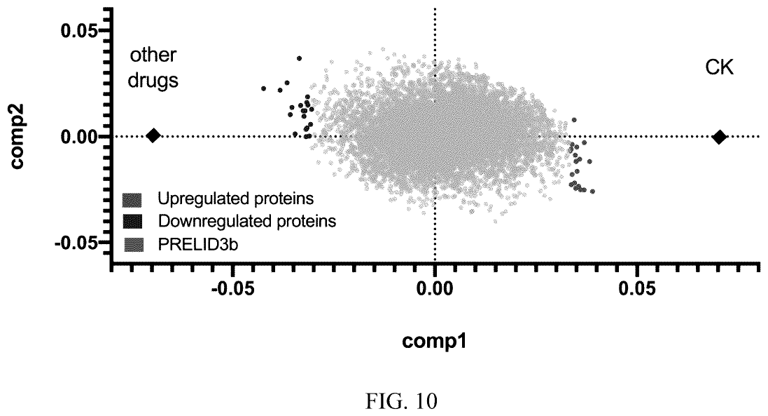

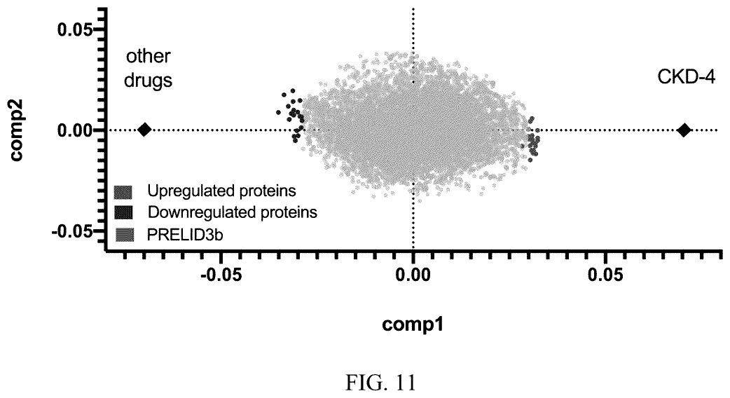

Two label free chemical proteomics, proteome integral stability alteration assay (PISA) and ProtargetMiner (PTM) are alternative strategies to study CK and CKD-4. PISA is a turbo-charged method development of thermal proteome profiling (TPP) which based on the principle that ligand induced stability of proteins is observed upon a temperature increase. PTM is used to contrast the proteome signature of CK and CKD-4 against the PTM database to reveal their mode of action. FIGS. 9A and 9B provide an overview of the PISA results in cells with CK and CKD-4, where data is shown as a volcano plot with the X-axis as log₂ ΔSₘ of drug over DMSO treatment and the Y-axis as log 10 p-value. In FIGS. 10 and 11 the PTM result of CK and CKD-4 in cells are compared to 9 other anti-cancer drugs in A549 cells in the database and is plotted for OPLS-DA analysis.

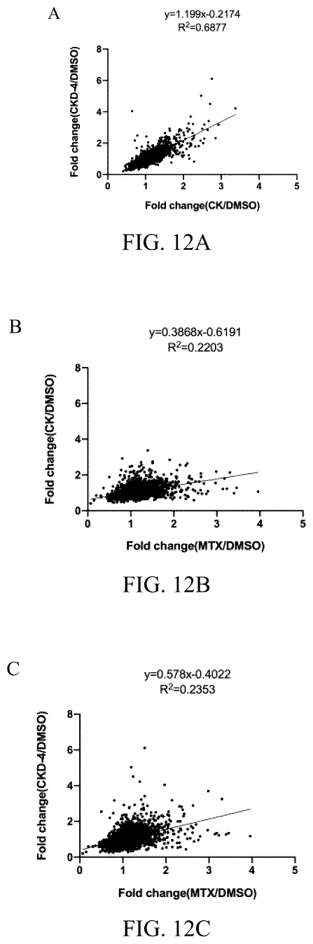

To investigate that CKD-4 has the same mechanism as CK after modification, protein expression profiles (ratio of drug over DMSO treatment) in PTM are compared. The expression profile of CKD-4 and CK correlate significantly with R² equals to 0.6877, as shown in FIG. 12A. In the contrast, CK and CKD-4 do not show significant correlation with methotrexate as both have a low R² value, shown in FIGS. 12B and 12C, respectively.

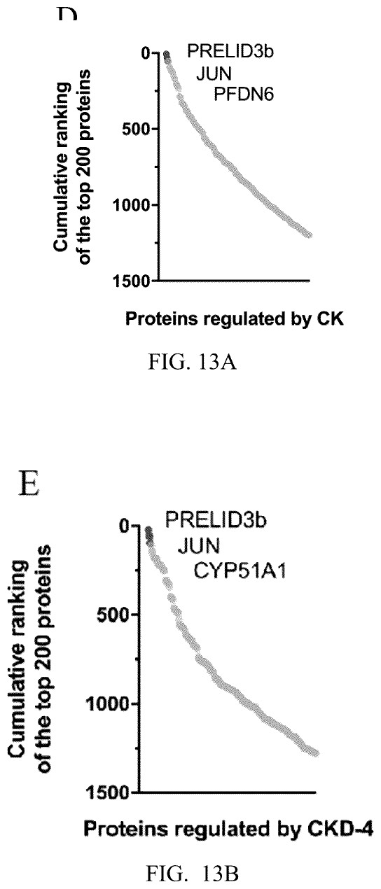

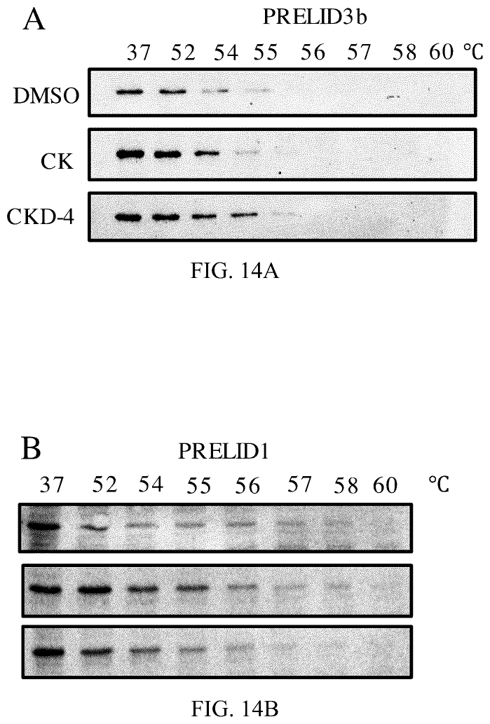

Most probable target candidate proteins are ranked in PISA based on the absolute value of log₂ ΔSₘ of drug relative to DMSO treatment and PTM ranking are based on the log₂ fold change of drug over DMSO treatment. The rankings of two experiments are summed to provide a list of candidate drug targets. PRELID3b and JUN stand out as the first and second candidate as indicated in FIGS. 13A and 13B. Hence, studies focus on PRELID3b. Interestingly, PRELID1 is stabilized by CK and CKD-4 in PISA although this protein is not identified in PTM experiments. The stabilization of PRELID3b and PRELID1 is confirmed using cellular thermal shift assay by western blotting, which showed thermal stability of both proteins by CK and CKD-4 upon treatment in A549 cell, as indicated in FIGS. 14A and 14B.

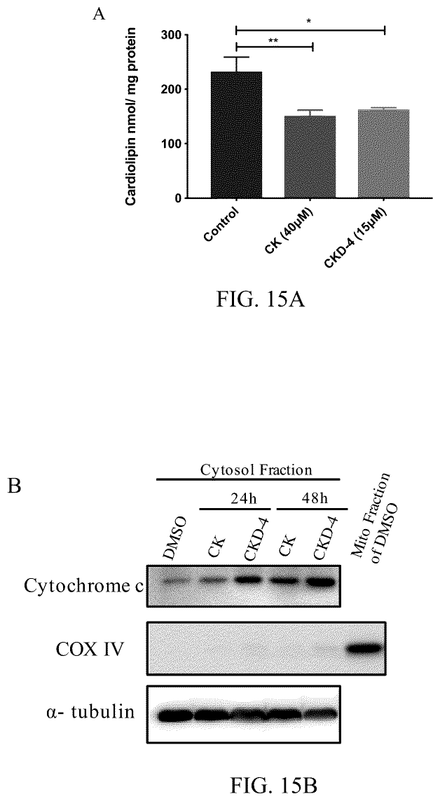

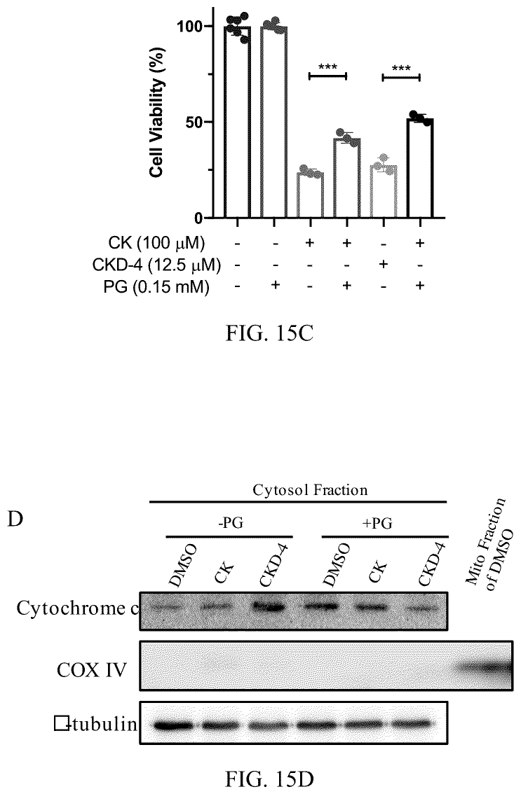

PRELID3b and PRELID1 are PRELI proteins. These proteins and TRIAP1 locate within the inter membrane space responsible for the regulation of phospholipid metabolism. Therefore, a cardiolipin probe is used to measure levels of mitochondrial cardiolipin after drug treatment. The amount of cardiolipin decreased significantly after a 24-hour treatment with either CK or CKD-4 in A549 cells is shown in FIG. 15A. A decreased level of cardiolipin in PRELI or TRIAP1 depleted cells appears to accelerate the release of cytochrome c, rendering cells vulnerable to apoptosis. Consistent with this phenomenon, after a 24-hour treatment with CK and CKD-4, the level of cytochrome c in cytosol is dramatically enhanced, as indicated in FIG. 15B. Cardiolipin is synthesized by a series of steps with phosphatidylglycerol (PG) as the precursor of last step catalyzing by cardiolipin synthase. To demonstrate the decrease of cardiolipin contributes to the cell apoptosis, PG is artificially supplied in the medium to restore levels of CL during treatment with CK and CKD-4. NBB assay shows that with supplement of PG, cell viability is almost two-fold restored upon treatment with 100 μM of CK and 12.5 μM of CKD-4 after 24 hours, respectively, and indicated in FIG. 15C. Reduced level of cytochrome c was released in the case of CKD-4 although this was not observed for CK, as indicated in FIG. 15D.

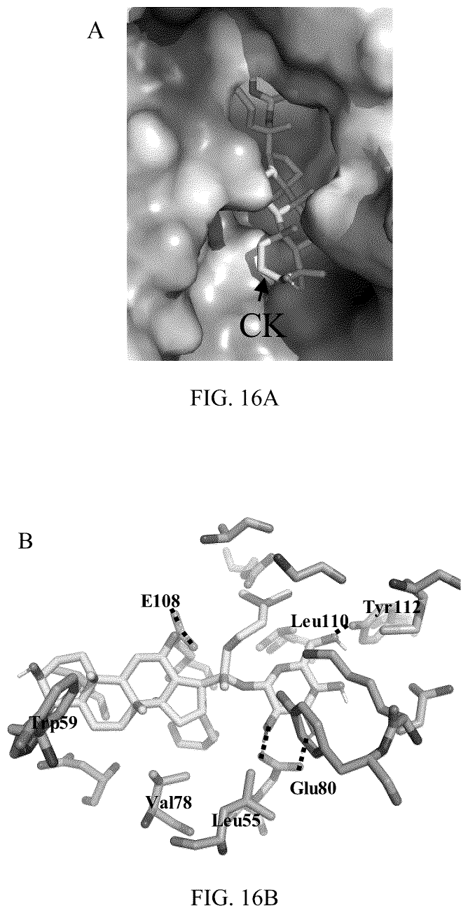

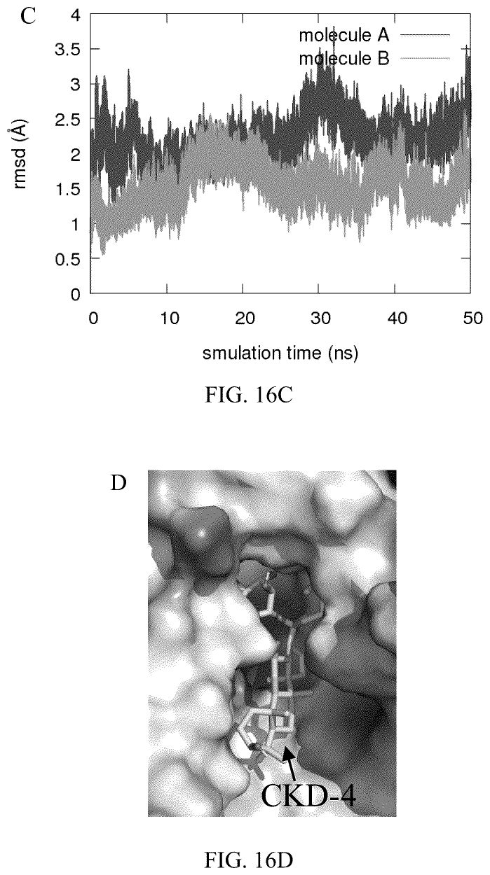

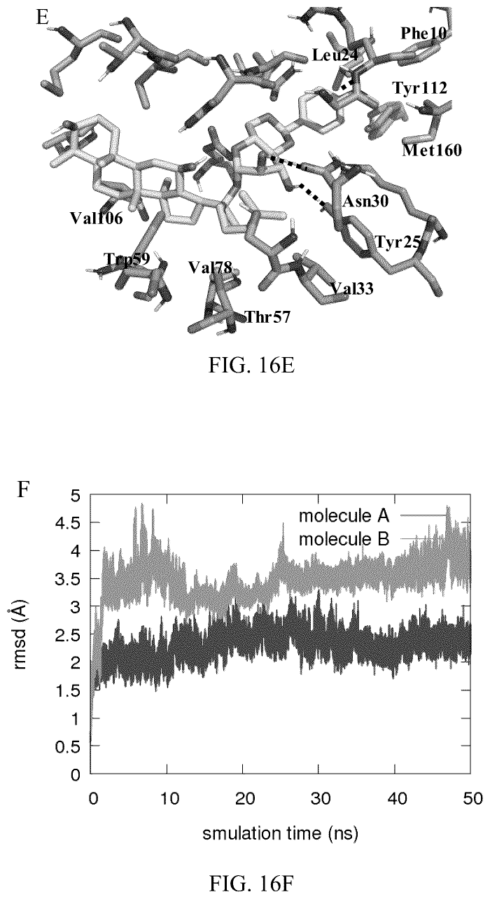

Computational docking and molecular dynamics simulations provide a model of interactions between CK or CKD-4 and PRELID3b. Based on the chemical similarity between CK and phosphatidylserine lipid, the lipid binding cavity of PRELID3b is assumed as the potential binding pocket of CK and CKD-4. For both compounds, top poses predicted by docking are subjected to 50 ns MD simulations for stability assessment, which is an efficient approach to identify correct binding pose and to refine docking predicted binding poses. The computational results are summarized in Table 4, below, and illustrated in FIGS. 16A through 16F.

TABLE 4
Docking result of CK and CKD-4 with PRELID3b

| | ΔG | ΔE_{vdw+HB} | ΔEₑₛ | ΔE_{desolv} | ΔEₜₒᵣₛᵢₒₙ | |
|---|---|---|---|---|---|---|
| | (kcal/ | (kcal/ | (kcal/ | (kcal/ | (kcal/ | K_{d} |
| | mol) | mol) | mol) | mol) | mol) | (μM) |
| CK | −6.55 | −15.13 | −1.19 | 6.89 | 3.88 | 15.84 |
| CKD-4 | −7.52 | −18.40 | −0.24 | 7.53 | 3.58 | 3.07 |

The CK molecules bound to PRELID3b dimer are quite stable as supported by the ligand rsmd profiles. The averaged rmsd values of the two CK molecules during the last 10 ns are 2.3 Å and 1.5 Å, respectively, which are under the generally accepted threshold of 2.5 Å for a stable binding. The PRELID3b/CKD-4 complex is also shown in FIGS. 16A and 16B. The averaged rmsd values of the last 10 ns for the two CKD-4 molecules are 2.4 Å and 3.7 Å, respectively. Regarding the stable poses of CK and CKD-4, AutoDock estimated binding energies are −6.55 kcal/mol and −7.52 kcal/mol, respectively. In terms of dissociation constants, the affinity of CKD-4 is 5-fold higher than that of CK.

In the lipid-binding pocket, the orientations of both CK and CKD-4 are similar to the PS lipid, as illustrated in FIGS. 16C and 16F. The hydrophilic glucose heads are buried in the deep cavity, while the hydrophobic gonane moiety attaches to the hydrophobic wall formed by residues Val33, Leu55, Thr57, Trp59, Thr76 and Va178. The major differences between CK and CKD-4 are in the locations of the glucose head and the alkene chain. The glucose head of CK is close to Glu80 and forms hydrogen bonds with the carboxylic oxygen atom of Glu80 and the phenol group of Tyr112. The alkene chain is buried under the 3 helix. In the case of CKD-4, however, the p-methoxybenzyl (PMB) protected glucose unit, instead of the alkene chain, is buried under 3. The PMB group is surrounded by residues Phe10, Lys27, Leu110, Tyr112, Leu124, Gln126 and residues of the 3 helix including Gly156, Arg157 and Met160. The hydroxyl groups on the glucose ring form hydrogen bonds with side chains of Tyr26 and Asn30. The alkene chain is attached to a hydrophobic pocket formed by residues Val33, Thr57, Trp59 and Va178.

Apparently, the large PMB group creates more hydrophobic contacts between CKD-4 and PRELID3b, leading to the enhanced binding affinity of CKD-4. This hypothesis is supported by the decomposition of the binding energies of both compounds. AutoDock score function estimates the binding energy by combining the van der Waals interactions, hydrogen bonds, electrostatic interactions, desolvation effect and the ligand torsional energy. By comparing these terms, the combination of van der Waals and hydrogen bonds (ΔEvdw+HB) is the major contribution (3.26 kcal/mol) to the binding energy difference. As shown in FIG. 16E, only two hydrogen bonds are found between CKD-4 and the protein, which is in contrast to the four hydrogen bonds between CK and the protein. Therefore, the van der Waals interactions between CKD-4 and PRELID3b exceed the loss of hydrogen bonds and thereby leading to increase the total binding energy.

To validate the docking analysis, biolayer interferometry (BLI) was employed to measure the interaction between PRELID3b and CK or CKD-4. The recombinant protein was purified by co-expression of PRELID3b with a hexa-histidine tag and the p53 regulated protein, TRIAP1 conjugated with maltose binding protein (MBP). The reported crystal structure of RELID3b is in complex with TRIAP1 and they form as a swapped dimer. PRELID3b is intrinsically unstable in the absence of TRIAP1 and easily degraded by mitochondrial protease. Maltose binding protein (MBP) is a necessary component to improve the solubility of the whole protein complex.

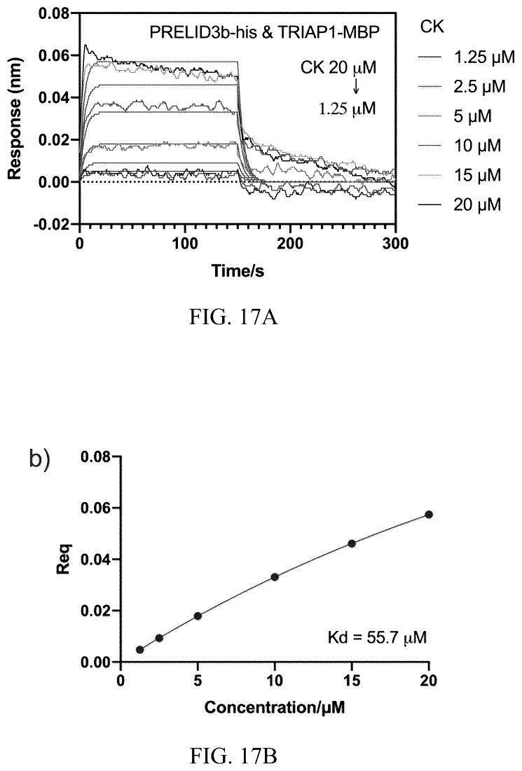

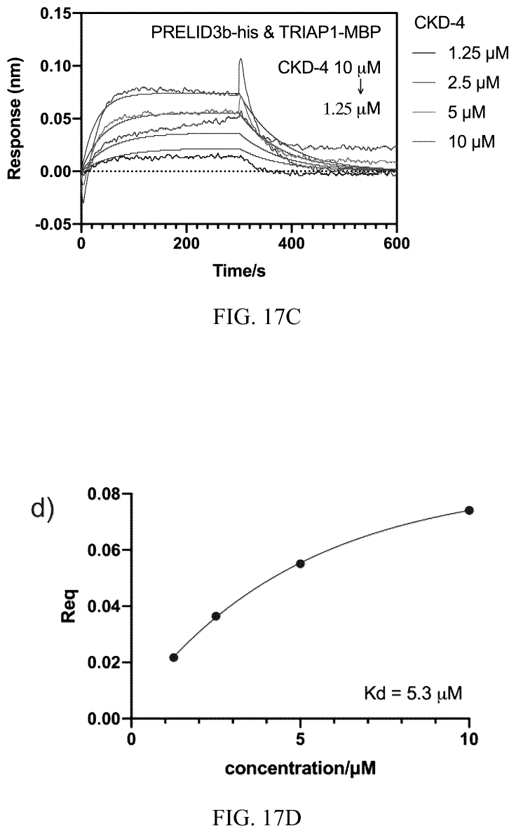

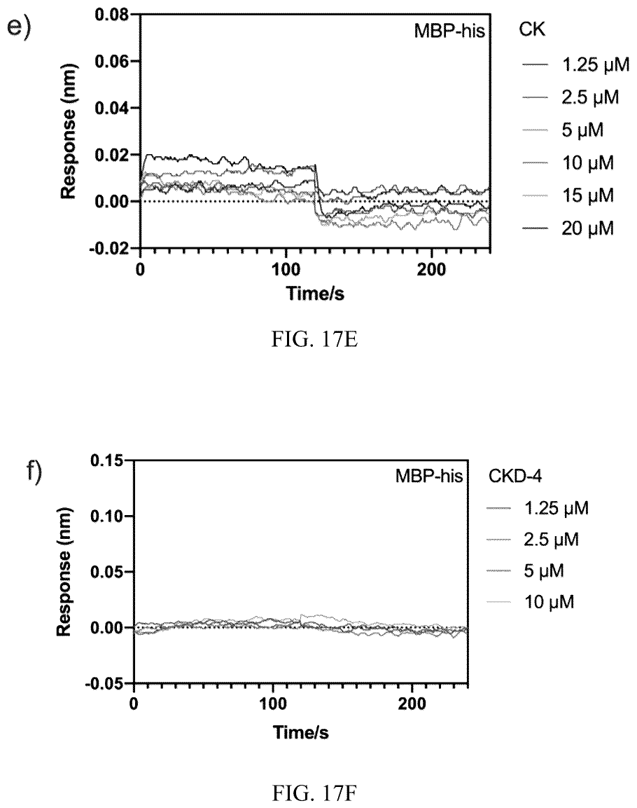

The sensor used is coated with Ni NTA so that proteins with the hexa-histidine tag could be immobilized on the sensor. After immobilization with protein, the sensor was incubated with the compounds at different concentrations in an ascending order. Both the stoichiometry of CK or CKD-4 with PRELID3b is in a 1:1 ratio but the kinetic modes for the two cases are different. CK interacts with PRELID3b with a fast on/off rate while the rate is slower in the case of CKD-4. This assay using PRELID3b and CKD-4 gave the Kd of 5.3 μM which is almost 7-fold smaller than the Kd (55.7 μM) between CK and PRELID3b, as shown in FIGS. 17A through D. In order to prove the interaction is not attributed to the existence of MBP, the interaction between MBP and CK or CKD-4 was examined. The result indicated that no does-dependent response occurs for either CK or CKD-4 as indicated in FIGS. 17E and 17F. The result is consistent with the docking analysis showing that the interaction between CKD-4 and PRELID3b is stronger than with CK.

### Materials and Methods

Synthesis of CKD-2

To a round bottom flask, p-toluenesulfonic acid (13.9 mg, 0.081 mmol) was added to a mixture of acetone and 2,2-dimethoxypropane (5:1, v/v, 20 mL) containing CK (50.3 mg, 0.081 mmol) under argon. The reaction was stirred at room temperature for 1.5 hours and the progress was monitored by TLC analysis. Triethylamine (1 mL) was added to quench the reaction. The mixture was concentrated, diluted with dichloromethane, and washed with water for multiple times. The organic fraction was dried with anhydrous MgSO₄, followed by filtration and concentration under reduced pressure. Flash column chromatography was used for purification yielding CKD-2 (3.8 mg, 0.0057 mmol, 7% yield). ¹H NMR (400 MHz, CDCl₃), δ ppm 5.29 (m, 1H), 4.57 (d, J=7.7 Hz, 1H), 3.84 (m, 2H), 3.65 (m, 3H), 3.39 (t, J=4.4 Hz, 1H), 3.21 (t, J=4.4 Hz, 2H), 1.68-0.78 (m, 30H). ¹³C NMR (600 MHz, CDCl₃), δ ppm 124.45, 99.75, 97.48, 84.49, 78.86, 74.55, 73.84, 72.83, 70.59, 67.17, 62.26, 55.79, 51.70, 51.42, 49.77, 47.95, 39.75, 38.91, 38.87, 37.06, 35.41, 34.71, 30.61, 30.44, 29.70, 29.08, 28.01, 27.38, 26.66, 25.71, 22.27, 21.62, 19.13, 18.25, 17.75, 16.94, 16.12, 15.71, 15.37. MS (ESI): m/z for C₃₇H₆₂O₈, calcd 634.4445 [M+Na]⁺ found 685.30.

### Synthesis of CKD-3

To a solution of 2,2-dimethoxy propane (20 mL) and CK (49.3 mg, 0.079 mmol), p-toluenesulfonic acid (13.6 mg, 0.079 mmol) was added under argon. The mixture was stirred overnight at room temperature and stopped by addition of triethylamine (1 mL). Dichloromethane was added to dilute the mixture and the mixture was washed with water. Anhydrous MgSO₄ was used to dry the mixture, followed by filtration and concentration under reduced pressure. Flash column chromatography purification finally yielded CKD-3 (11.3 mg, 0.016 mmol, 20.3%). ¹H NMR (400 MHz, CDCl₃), δ ppm 5.15 (m, 1H), 4.91 (d, J=7.6 Hz, 1H), 4.06 (m, 1H), 3.95 (m, 1H), 3.63 (m, 1H), 1.70-0.78 (m, 36H). ¹³C NMR (400 MHz, CDCl₃), δ ppm 125.30, 109.63, 108.87, 94.87, 79.28, 78.79, 78.53, 78.12, 76.72, 76.36, 70.87, 67.61, 55.93, 50.77, 50.33, 48.49, 48.37, 39.71, 39.13, 38.97, 37.28, 35.90, 34.79, 32.73, 29.72, 28.71, 28.04, 27.40, 26.86, 26.68, 25.78, 25.53, 25.44, 24.71, 22.09, 18.28, 17.74, 16.35, 16.26, 15.45, 15.35. HRMS(ESI): m/z for C₄₂H₇₀O₈, calcd 702.5071 [M+Na]⁺ found 725.4977.

### Synthesis of CKD-4

To a round bottom flask, p-anisaldehyde (5 mL) and boron trifluoride diethyl etherate (100 L) were added and suspended in methanol (80 mL). The resultant mixture was stirred and heated under reflux overnight. Triethylamine (10 mL) was used to quench reaction. Ether was used to extract the product, followed by washing with K₂CO₃ solution. Anhydrous MgSO₄ was used to dry the mixture, followed by filtration and concentration under reduced pressure. Flash column chromatography purification finally yielded 1-(dimethoxymethyl)-4-methoxybenzene.

To a suspension of CK (50 mg, 0.08 mmol) in dry dichloromethane (5 mL), 1-(dimethoxymethyl)-4-methoxybenzene (2 mL) and boron trifluoride diethyl etherate (6 μL) were added under argon. The resultant mixture was stirred at room temperature overnight and TLC was employed to monitor the progress until all CK was consumed. Triethylamine (1 mL) was added to stop the reaction. Dichloromethane was used to extract the resultant product, followed by washing with K₂CO₃ solution. Anhydrous MgSO₄ was used to dry the mixture, followed by filtration and concentration under reduced pressure. Flash column chromatography purification finally yielded CKD-4 (19.3 mg, 0.026 mmol, 32.5% yield). ¹H NMR (400 MHz, CDCl₃), δ ppm 7.43 (d, J=8.7 Hz, 2H), 6.89 (d, J=8.8 Hz, 2H), 5.49 (s, 1H), 5.12 (m, 1H), 4.64 (d, J=7.7 Hz, 1H), 4.26 (m, 1H), 3.57 (m, 2H), 3.44 (m, 2H), 1.80-0.77 (m, 24H). ¹³C NMR (600 MHz, CDCl₃), δ ppm 160.18, 131.63, 129.54, 127.60, 124.47, 113.64, 101.70, 97.40, 84.51, 80.16, 78.85, 74.43, 73.54, 70.57, 68.76, 66.19, 55.80, 55.30, 51.65, 51.44, 49.79, 48.00, 39.47, 38.89, 37.04, 35.39, 34.71, 30.58, 30.36. HRMS(ESI): m/z for C₄₄H₆₈O₉, calcd 740.4863 [M+Na]⁺ found 763.4765.

### Synthesis of CKD-5

Methanol (60 mL), benzaldehyde (5 mL) and boron trifluoride diethyl etherate (2 mL) were combined, stirred, and heated under reflux overnight. Triethylamine (10 mL) was added to quench the reaction. Ether was used to extract the resultant product, followed by washing with K₂CO₃ solution. Anhydrous MgSO₄ was used to dry the mixture, followed by filtration and concentration under reduced pressure. Flash column chromatography purification finally yielded (dimethoxymethyl)benzene.

To a suspension of CK (31.6 mg, 0.05 mmol) in dry dichloromethane (5 mL), dimethoxymethyl)-benzene (1 mL) and boron trifluoride diethyl etherate (6 μL) were added under argon. The resultant mixture was stirred at room temperature overnight and TLC was used to monitor the reaction. The reaction was stopped with triethylamine (1 mL). Dichloromethane was used to extract the resultant product, followed by washing with K₂CO₃ solution. Anhydrous MgSO₄ was used to dry the mixture, followed by filtration and concentration under reduced pressure. Flash column chromatography purification finally yielded CKD-5. ¹H NMR (400 MHz, CDCl₃), δ ppm 7.50-7.47 (m, 2H), 7.36-7.34 (m, 3H), 5.53 (s, 1H) 5.10 (t, J=6.7 Hz, 1H), 4.64 (d, J=7.7 Hz, 1H), 4.27-4.25 (m, 1H), 3.81-3.77 (m, 2H), 3.59-3.54 (m, 2H). MS(ESI): m/z for C₄₃H₆₆O₈, calcd 710.4758 [M+Na]⁺ found 733.40.

All patents, patent applications, provisional applications, and publications cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Wong A S, Che C M, Leung K W. Recent advances in ginseng as cancer therapeutics: a functional and mechanistic overview. Nat Prod Rep. 32, 256-72 (2015).
C. Gridelli et al., Non-small-cell lung cancer. Nature Reviews Disease Primers 1, 15009 (2015).
L. Zhou, Z. K. Li, C. Y. Li, Y. Q. Liang, F. Yang, Anticancer properties and pharmaceutical applications of ginsenoside compound K: A review. Chem Biol Drug Des 99, 286-300 (2022).
B. H. Han et al., Degradation of ginseng saponins under mild acidic conditions. 44, 146-149 (1982).
D.-H. J. J. o. g. r. Kim, Gut microbiota-mediated pharmacokinetics of ginseng saponins. 42, 255-263 (2018).
L. Chen et al., Ginsenoside compound K sensitizes human colon cancer cells to TRAIL-induced apoptosis via autophagy-dependent and -independent DR5 upregulation. Cell Death Dis 7, e2334 (2016).
K. Zhang, Y. Li, Effects of ginsenoside compound K combined with cisplatin on the proliferation, apoptosis and epithelial mesenchymal transition in MCF-7 cells of human breast cancer. Pharm Biol 54, 561-568 (2016).
Z. Z. Zheng et al., Compound K-induced apoptosis of human hepatocellular carcinoma MHCC97-H cells in vitro. Oncol Rep 32, 325-331 (2014).
C. Li et al., Ginsenoside metabolite compound K induces apoptosis and autophagy in non-small cell lung cancer cells via AMPK-mTOR and JNK pathways. Biochem Cell Biol 97, 406-414 (2019).
H. F. Chen et al., Ginsenoside compound K inhibits growth of lung cancer cells via HIF-1α-mediated glucose metabolism. Cell Mol Biol (Noisy-le-grand) 65, 48-52 (2019).
Y. Li et al., Ginsenoside metabolite compound K enhances the efficacy of cisplatin in lung cancer cells. J Thorac Dis 7, 400-406 (2015).
S. Chae et al., Effect of compound K, a metabolite of ginseng saponin, combined with gamma-ray radiation in human lung cancer cells in vitro and in vivo. J Agric Food Chem 57, 5777-5782 (2009).
S. Ziegler, V. Pries, C. Hedberg, H. Waldmann, Target identification for small bioactive molecules: finding the needle in the haystack. Angew Chem Int Ed Engl 52, 2744-2792 (2013).
M. M. Savitski et al., Tracking cancer drugs in living cells by thermal profiling of the proteome. Science 346, 1255784 (2014).
A. Chernobrovkin, C. Marin-Vicente, N. Visa, R. A. Zubarev, Functional Identification of Target by Expression Proteomics (FITExP) reveals protein targets and highlights mechanisms of action of small molecule drugs. Sci Rep 5, 11176 (2015).
R. Jafari et al., The cellular thermal shift assay for evaluating drug target interactions in cells. Nat Protoc 9, 2100-2122 (2014).
M. Gaetani et al., Proteome Integral Solubility Alteration: A High-Throughput Proteomics Assay for Target Deconvolution. J Proteome Res 18, 4027-4037 (2019).
A. A. Saei et al., ProTargetMiner as a proteome signature library of anticancer molecules for functional discovery. Nat Commun 10, 5715 (2019).
D. C. Wallace, Mitochondria and cancer. Nature Reviews Cancer 12, 685-698 (2012).
N. Stepanyants et al., Cardiolipin's propensity for phase transition and its reorganization by dynamin-related protein 1 form a basis for mitochondrial membrane fission. Mol Biol Cell 26, 3104-3116 (2015).
J. Dudek, Role of Cardiolipin in Mitochondrial Signaling Pathways. Front Cell Dev Biol 5, 90 (2017).
S. T. Ahmadpour et al., the Mitochondrial Signature Lipid: Implication in Cancer. Int J Mol Sci 21 (2020).
X. Miliara et al., Structural insight into the TRIAP1/PRELI-like domain family of mitochondrial phospholipid transfer complexes. EMBO Rep 16, 824-835 (2015).
B. Y. Kim et al., Effects of PRELI in Oxidative-Stressed HepG2 Cells. Ann Clin Lab Sci 45, 419-425 (2015).
A. E. Gillen et al., Alternative Polyadenylation of PRELID1 Regulates Mitochondrial ROS Signaling and Cancer Outcomes. Mol Cancer Res 15, 1741-1751 (2017).
T. MacVicar et al., Lipid signalling drives proteolytic rewiring of mitochondria by YME1L. Nature 575, 361-365 (2019).
J. P. Medema, Cancer stem cells: the challenges ahead. Nat Cell Biol 15, 338-344 (2013).
X. Miliara et al., Structural determinants of lipid specificity within Ups/PRELI lipid transfer proteins. Nat Commun 10, 1130 (2019).
P. Mishra et al., Metabolic regulation of mitochondrial dynamics. J Cell Biol 212, 379-387 (2016).
T. Ban et al., Molecular basis of selective mitochondrial fusion by heterotypic action between OPA1 and cardiolipin. Nat Cell Biol 19, 856-863 (2017).
M. J. Aaltonen et al., MICOS and phospholipid transfer by Ups2-Mdm35 organize membrane lipid synthesis in mitochondria. J Cell Biol 213, 525-534 (2016).
K. Quan et al., Rapid preparation of rare ginsenosides by acid transformation and their structure-activity relationships against cancer cells. Sci Rep 5, 8598 (2015).
K. G. CSH Tan et al., Thermal proximity coaggregation for system-wide profiling of protein complex dynamics in cells. Science 359, 1170-1177 (2018).
K. V. Huber et al., Proteome-wide drug and metabolite interaction mapping by thermal-stability profiling. Nat Methods 12, 1055-1057 (2015).
S. Sridharan et al., Proteome-wide solubility and thermal stability profiling reveals distinct regulatory roles for ATP. Nature Communications 10, 1155 (2019).
J. X. Huang et al., High throughput discovery of functional protein modifications by Hotspot Thermal Profiling. Nat Methods 16, 894-901 (2019).
I. Becher et al., Pervasive Protein Thermal Stability Variation during the Cell Cycle. Cell 173, 1495-1507. e1418 (2018).
C. Potting et al., TRIAP1/PRELI complexes prevent apoptosis by mediating intramitochondrial transport of phosphatidic acid. Cell Metab 18, 287-295 (2013).
W. C. Potting et al., Regulation of mitochondrial phospholipids by Ups1/PRELI-like proteins depends on proteolysis and Mdm35. EMBO J. 29, 2888-2898 (2010).
J. W. Ma et al., The role of mitochondrial dynamics in human cancers. Am J Cancer Res 2020; 10(5):1278-1293 10, 1278-1293 (2020).
G. R. Anderson et al., Dysregulation of mitochondrial dynamics proteins are a targetable feature of human tumors. Nat Commun 9, 1677 (2018).
M. R. McKeller et al., Vital function of PRELI and essential requirement of its LEA motif. Cell Death Dis 1, e21 (2010).
D. C. Chan, Fusion and fission: interlinked processes critical for mitochondrial health. Annu Rev Genet 46, 265-287 (2012).
C. R. Chang, et al., AMP-dependent protein kinase phosphorylation of Drp1 regulates its GTPase activity and mitochondrial morphology. J Biol Chem 282, 21583-21587 (2007).

We claim:

1. An anticancer compound comprising a derivative of 20-O-β-(D-glucopyranosyl)-20(S)-protopanaxadiol (CK) having at least one glucose hydroxyl group replaced with at least one acetal or ketal group, wherein the acetal group provides a hydrophobic portion that enhances binding to a mitochondrial membrane protein, PRELID3b, capable of assembling a lipid transfer complex with TRIAP1 in mitochondria and wherein the cytotoxicity of the CK derivative (CKD) is greater than the cytotoxicity of CK.

2. The anticancer compound according to claim 1, wherein the CKD comprises: 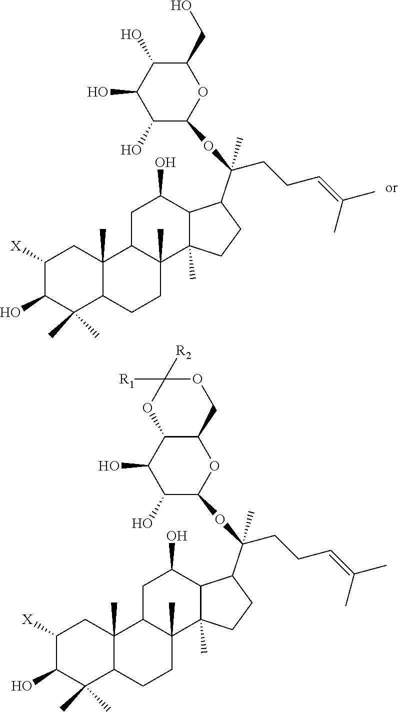
where X is H or F, R₁ is independently H, F or methyl and R₂ is independently F, methyl or an unsubstituted, monosubstituted, or disubstituted aromatic group.

3. The anticancer compound according to claim 2, wherein R₁ is H and R₂ is —C₆HₓR₅₋ₓ where x is 3 to 5 and R is independently selected from methoxy, nitro, trifluoromethyl, and trifluoromethoxy.

4. The anticancer compound according to claim 2, wherein R₁ is H and R₂ is —C₆H₄OCH₃ and wherein the OCH₃ is in the para, meta, or ortho position.

5. The anticancer compound according to claim 2, wherein x is 4 and R₁ is H and R₂ is p-methoxyphenyl (CKD-4).

6. The anticancer compound according to claim 2, wherein x is 4 and R₁ is H and R₂ is phenyl (CKD-5), o-methoxyphenyl (CKD-6), m-methoxyphenyl (CKD-7), p-nitrophenyl (CKD-8), p-trifluoromethyl (CKD-9) or p-trifluoromethoxyphenyl (CKD-10).

7. The anticancer compound according to claim 2, wherein the glucose hydroxyl groups form a ketal of 2,2-propane and a ketal of 2, 2-difluoromethane.

8. An anticancer medicament, comprising at least one anticancer compound according to claim 1.

9. The anticancer medicament according to claim 8, wherein the anticancer compound has the structure: 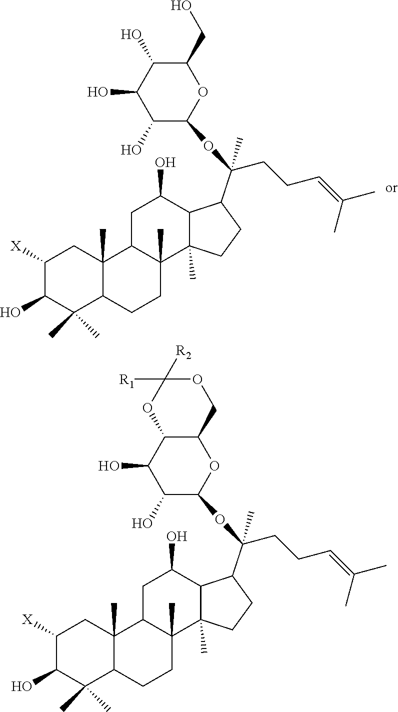
where X is H or F, R₁ is independently H, F or methyl and R₂ is independently F, methyl or an unsubstituted, mono substituted, or disubstituted aromatic group.

10. The anticancer medicament according to claim 9, wherein R₁ is H and R₂ is —C₆HₓX₅₋ₓ where x is 3 to 5 and X is independently selected from methoxy, nitro, trifluoromethyl, and trifluoromethoxy.

11. The anticancer medicament according to claim 9, wherein R₁ is H and R₂ is —C₆H₄OCH₃ and wherein the OCH₃ is in the para, meta, or ortho position.

12. The anticancer medicament according to claim 9, wherein R₁ is H and R₂ is p-methoxyphenyl (CKD-4).

13. The anticancer medicament according to claim 9, wherein R₁ is H and R₂ is phenyl (CKD-5), o-methoxyphenyl (CKD-6), m-methoxyphenyl (CKD-7), p-nitrophenyl (CKD-8), p-trifluoromethyl (CKD-9), or p-trifluoromethoxyphenyl (CKD-10).

14. A method of preparing an anticancer compound according to claim 1, comprising:
providing 20-O-β-(D-glucopyranosyl)-20(S)-protopanaxadiol (CK);
providing an aldehyde, acetal, ketone, or ketal comprising reagent;
combining the CK and the reagent to form a CK derivative (CKD); and
isolating the CKD.

15. The method according to claim 14, wherein the ketal or the acetal comprising reagent is 2,2-dimethoxypropane or dimethoxydifluoromethane.

16. The method according to claim 14, wherein the acetal comprising reagent is 1-(dimethoxymethyl)-4-methoxybenzene.

17. The method according to claim 14, wherein the acetal comprising reagent is dimethoxymethyl-benzene, 1-(dimethoxymethyl)-3-methoxybenzene, 1-(dimethoxymethyl)-2-methoxybenzene, 1-(dimethoxymethyl)-4-nitrobenzene, 1-(dimethoxymethyl)-4-trifluoromethylbenzene, or 1-(dimethoxymethyl)-4-trifluoromethoxylbenzene.

18. The method according to claim 14, further comprising adding a solvent.

19. The method according to claim 14, further comprising adding a catalyst with the CK and the reagent.

20. The method according to claim 14, wherein isolating the CKD comprises performing chromatography.
